(19) United States
(12) Patent Application Publication
(10) Pub. No.: US 2015/0210979 A1
(43) Pub. Date: Jul. 30, 2015

(54) SCAFFOLD-FREE TISSUE ENGINEERING USING FIELD INDUCED FORCES

(71) Applicant: NORTHROP GRUMMAN SYSTEMS CORPORATION, FALLS CHURCH, VA (US)

(72) Inventor: Sameh S. Wanis, Alexandria, VA (US)

(73) Assignee: NORTHROP GRUMMAN SYSTEMS CORPORATION, FALLS CHURCH, VA (US)

(21) Appl. No.: 14/165,085

(22) Filed: Jan. 27, 2014

(51) Int. Cl. C12N 5/00; C12M 3/00

(52) U.S. Cl. CPC C12N 5/0062; C12M 21/08; C12N 2527/00

(57) ABSTRACT

A system and method for providing tissue regeneration without the use of scaffolds. The system includes a vessel that contains a fluid suitable for enhancing the tissue regeneration process. An acoustic transducer is provided at one end of the vessel and a reflector is provided at an opposite end of the vessel. The transducer provides an acoustic signal that creates standing acoustic fields in the vessel that confine human cells within the fluid into a plurality of cell sheets. A system of electrodes provides dielectrophoretic forces within the vessel to create cellular chain arrays to provide vascularization for the tissue.

### BACKGROUND

1. Field

This invention relates generally to a system and method for providing tissue regeneration without the use of scaffolds and, more particularly, to a system and method for providing tissue regeneration that employs acoustic fields to confine human cells into sheets and electric fields to form cell assemblies into chains for vascularization.

2. Discussion

It is known in the art to use acoustic forces and electric forces to manipulate and confine particles. Applications for using these forces have varied, but mainly focus on using fields for non-contact levitation and positioning of particles.

The first observation of time-averaged radiation pressure was made in 1866 when dust particles were observed to be collected at the pressure nodes of standing fields in circular ducts. Forces due to acoustic radiation pressure were first measured in 1903. Work in this area theoretically addressed both traveling and standing plane fields incident on a rigid sphere and showed that the force due to radiation pressure is proportional to the sound field intensity for traveling fields and proportional to its gradient in standing fields. Using air as a host fluid levitation in a one-dimensional environment requires powerful acoustic sources placed along the vertical axis, usually in opposition or in a source/reflector configuration. With sufficiently high sound levels, forces induced on particles cause the particles to move towards the nodes. The speakers and reflector surfaces can be tailored to ensure that there is only one stable point of minimum acoustic force potential for the particle to occupy.

Acoustic levitation has been used in several applications. For example, this principle has been used to study material properties in a microgravity environment where the same principals of acoustic levitation to control position and deform a droplet in a gaseous fluid host have been employed. A material transport system has been developed in the art where multiple modes were excited simultaneously. As the relative intensities of the modes were varied, the stable location of the particle, which lies between the potential wells of each mode, was controlled.

The idea that light can exert forces on neutral matter was introduced as far back as Kepler and Newton and was confirmed by Maxwell's equations for the propagation of electric fields. A plane electric wave impinging on a perfectly absorbing flat plate will exert a net radiation pressure, P=Q^{I}/_{c}, where I is the beam intensity, Q is 1 for perfectly absorbing plates and 2 for perfectly reflecting plates, and c is the speed of light. For a perfectly reflective surface the change in momentum is doubled and therefore the pressure is doubled (Q=2). Maxwell calculated the forces to be extremely small for conventional light sources. With the advent of the laser several experiments have demonstrated the capability of manipulating micro-meter sized particles using the momentum of laser light.

Most of the work relating to electric radiation pressure has been limited to the infrared-optical regime in the context of “optical tweezers.” Optical tweezers have the ability to manipulate and trap microscopic particles via the interaction between optic fields, i.e., electric fields, and matter. Starting with demonstrations of using radiation pressure to guide and trap particles, optical tweezers began to gain popularity by the breakthrough that a focused light beam attracts a small particle with an index of refraction higher than the host medium towards its beam focus. The wide range of use of optical tweezers is evident from its use in trapping and cooling neutral atoms to manipulating single living cells.

Optical tweezers have also been used to measure elastic properties of cells and molecules. It has shown that a pair of pig-tailed optical fibers can be used to utilize both scattering and gradient forces to trap particles in the gap between the fiber ends. This was later used to stretch soft biological samples due to the sample having a higher index of refraction than the surrounding medium. Laser tweezers composed of a pair of pig-tailed fiber optics have been used to control trapped particles of various sizes. Tailoring of the light field is mentioned as a means of enhancing the capability of optical tweezers in molecular level manufacturing.

Acoustic fields have been used for sorting cells based on their different sizes and types, and have also been investigated for their effect on the tissue regeneration process.

For certain types of large wounds, such as, for example, battle field injuries, burns, etc., tissue regeneration is often necessary for the wound to properly heal. Tissue regeneration involves a set of intricate events that have to take place at the right time and place. Time scales range from seconds to weeks and length scales range from 1 μm to 10 cm, which makes tissue engineering a long way from being commercially available as off-the shelf products.

The current state-of-art of tissue regeneration includes providing a cell scaffold, typically a sponge-like bioactive and biodegradable material, as a support where the scaffold is seeded with cells from the patient to allow the cells to replicate in a three-dimensional manner and eventually form tissue. Scaffolds are needed to provide both physical and biochemical cues for cells to differentiate and assemble into a three-dimensional configuration. The scaffold is placed in a specialized solution including the proper chemicals and nutrients necessary to induce tissue regeneration from the cells that are in the scaffold, where the scaffold acts as a support surface that allows the cells to attach thereto. Seeding of the scaffold allows growth factors to reach the cells once attached to the scaffold and helps induce cell differentiation and tissue growth. Once enough tissue is regenerated within and around the scaffold, the combined scaffold and tissue are surgically implanted into the patient, where the scaffold then degrades overtime and dissolves into the patient's body.

The process for making a scaffold for tissue generation is very time consuming and each scaffold needs to be tailored to the specific patient. Because the scaffold needs to be custom made for each patient and the patient body needs to dissolve the scaffold once it has been surgically attached, using a scaffold for this purpose has a number of obvious drawbacks. Further, sometimes the body's immune system rejects the scaffold, because despite being biodegradable, the body's immune system is most likely heavily burdened at this point by the loss of tissue suffered.

### BRIEF DESCRIPTION OF THE DRAWING

FIG. 1 is a schematic-type view of a bioreactor system for regenerating tissue in a scaffoldless environment.

### DETAILED DESCRIPTION OF THE EMBODIMENTS

The following discussion of the embodiments of the invention directed to a system and method for providing tissue regeneration using acoustic fields to contain cells without the need for a scaffold is merely exemplary in nature, and is in no way intended to limit the invention or its applications or uses.

A small neutral particle in the presence of a wave will scatter a portion of that wave due to the impedance mismatch between the particle material and the host medium. Any type of wave motion can thus induce a force on a particle in its path. If the particle is small compared to the wavelength by about one order of magnitude, then the radiated field can be approximated by an electric dipole in an electric field and a combination of a monopole and dipole in an acoustic field. The fact that the particles behave as dipoles or monopoles in the field greatly reduce the mathematics to analytical solutions. The forces on a single particle in an acoustic field is given by:

$$F = \frac{5}{8} V ( \frac{5 p - 2 {p}_{o}}{2 p - {p}_{o}} - \frac{{p}_{o} {c}_{o}^{2}}{{pc}^{2}} ) ∇ ( \frac{{p}_{o}^{2}}{{p}_{o} {c}_{o}} ) . \qquad ( 1 )$$

And the forces on a particle in an electric field is given by:

$$F = \frac{3}{4} V ( \frac{ɛ - {ɛ}_{o}}{ɛ + 2 {ɛ}_{o}} ) ∇ ( {ɛ}_{o} {E}_{o}^{2} ) . \qquad ( 2 )$$

The present invention proposes employing acoustic fields and electric fields for inducing physical forces onto cells to produce three-dimensional tissue for treating wounded patients who need regenerated tissue. Acoustic and electric forces can be used to manipulate cells in a solution and shape them into predetermined geometries. This could serve as a scaffold-less bioreactor since no contact with the cells is necessary. Growth factors would reach the cells more readily since there is no scaffold obstructing the flow. The main goals of the invention are to increase the speed of the process for forming tissue and eliminate the need for a scaffold. Cells will collect into predetermined shapes by controlling the field distribution and the field type. Acoustic fields are proposed for creating cell sheets (surfaces) and electric fields are proposed for creating linear cell arrays (chains). Cells respond to field gradients due to the difference in impedance from their host medium. By carefully selecting and tuning the fields, the cells can be assembled and stably configured into complex three-dimensional geometries without the need to use a scaffold material. The rapid formation and sequential nature for “scaffold-less” assembly of cells should facilitate more rapid and complex, yet spatially organized, tissue structures than can be currently achieved by conventional tissue engineering techniques. In addition to being non-contact this process is also parallel in that the cells all respond to the field at the same time depending upon their respective positions within the field.

Forces induced on the cells will serve to speed up cell transport as well as to hold the cells in place for the required time needed for cell to cell adhesion to take place. As long as the field is on and no other competing forces dominate, the cells will stay in their original position. Higher frequency electric fields (optical fields), such as those produced by lasers, may be used to increase the speed of the cell adhesion process without affecting the cell assembly. Interaction between the primary acoustic shaping field and the adhesive field (lasers) is negligible as long as there is a large separation in wavelengths.

The cells will be placed in a bioreactor in a non-specific distribution in a fluid media, which includes necessary growth factors to keep the cells alive. Acoustic fields, electric fields, or a combination thereof, are used to create a potential energy landscape inside the bioreactor whereby cells collect at the wave or field minima's. A set of standing fields is produced when the bioreactor is excited at one of its resonant frequencies. The location and shape of these potential minima's is externally controlled by adjusting the field frequency and/or cavity shape. The end goal is to achieve: (a) faster bioreactors, (b) scaffold-less bioreactors, and (c) contamination free due to non-contact with bioreactor walls.

As discussed herein, human cells suspended in a fluid media are assembled at predetermined locations using non-contact field-induced forces for tissue regeneration. Holding the assembled cells in three-dimensional configurations for an extended period of time enables the cells to form a natural extra cellular matrix (ECM) where eventually tissue will form. The resulting process is a faster method for tissue engineering and the elimination of a custom scaffold that is the current state of the art. The use of multiple field frequencies enables the collection of individual cells into specific three-dimensional surfaces and movement of the formed tissue can be provided at lower frequencies. Thus, both the individual cells and formed tissue are manipulated using the field-induced forces. This enables multiple surfaces to be brought together for multi-cell type tissues to be formed. The integration of microfluidics to enable specific nutrients to be delivered to the proper locations at pre-specified times enables the viability of the cells to remain high.

As discussed, the present invention solves two major problems in tissue engineering and generation, namely, the elimination of a physical scaffold, and increasing the speed of the alignment of individual cells into the desired three-dimensional geometry. The first problem is associated with the complexity of constructing custom scaffolds, the problems associated with scaffold acceptance by the host patient, and the limitations posed by the flow of nutrients and waste removal to and from cells, where scaffolds must provide a delicate balance between being porous enough to allow nutrients to flow to the cells and waste to flow out and yet rigid enough to maintain the desired three-dimensional geometry and not get clogged up by cells going through its pores. The second problem deals with the fact that current tissue regeneration technologies rely on diffusive forces to drive cells to their predetermined locations, which is a small force in comparison to field-induced forces and is thus a slower process. Any time saved in the tissue regeneration procedure amounts to a greater chance for the patient to survive tissue and blood loss.

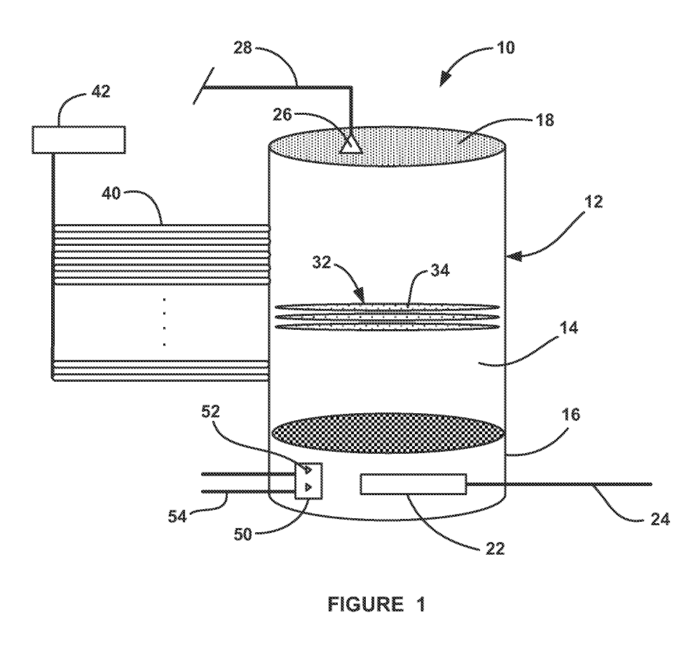

FIG. 1 is an illustration of a tissue regeneration bioreactor system 10 including a vessel 12 filled with a fluid 14 having suitable growth factors, known to those skilled in the art, for tissue regeneration. The vessel 12 sits on a base portion 16 and a top end of the vessel 12 includes a reflector 18. The fluid 14 is provided to the vessel 12 through a tube 28 coupled to a valve 26 at the top end of the vessel 12 where the reflector 18 is located. A transducer 22 is provided in the base portion 16 and is coupled to a power line 24. The transducer 22 is intended to represent any suitable device that can generate an acoustic signal at a desired frequency that will be reflected off of the reflector 18. The transducer 22 can be a tunable broadband transducer so that the particular acoustic signal generated within the vessel 12 can be selectively tuned within a predetermined frequency range. The length of the vessel 12 and the frequency of the acoustic signal is selectively provided to generate standing fields within the vessel 12 when reflected off of the reflector 18.

Cells 34 are provided in the fluid 14 either before the fluid 14 is put in the vessel 12, or otherwise as discussed below, and is interspersed therein in a non-specific configuration. When the transducer 22 is turned on, the standing fields cause the cells 34 to form a plurality of separated cell sheets 32, where a separate sheet 32 is formed at the nulls of the standing fields consistent with the discussion above. Typically, each of the sheets 32 has a thickness of one cell. As the cells 34 are held in the sheets 32, the cells 34 begin to interact forming an ECM where the once separated sheets 32 grow together to form tissue of a desired thickness. The more cells 34 that are in the vessel 12 the greater the number of the sheets 32 that will form. The greater the number of the sheets 32, the greater the thickness of the tissue.

The tissue regeneration process can be enhanced so that the type of tissue being generated and the speed at which the tissue is generated can be increased. To accomplish this, the system 10 includes a plurality of microfluidic tubes 40 that are strategically positioned at several locations along the length of the vessel 12 and are selectively open thereto so that different types of nutrients and other materials can be delivered to the vessel 12 at different locations within the vessel 12 and at different times to bio-engineer the tissue regeneration process. As mentioned above, the cells 34 can be delivered to the vessel 12 with the fluid 14 through the tube 28. Alternatively, the cells 34 can be delivered to the vessel 12 through the microfluidic tubes 40 after the fluid 14 is within the vessel 12. In this embodiment, different types of the tissue cells 34 can be delivered to the vessel 12 at different levels within the vessel 12. When the transducer 22 is operational, providing the different types of the tissue cells 34 to the vessel 12 at the different locations within the vessel 12 through the microfluidic tubes 40 causes the sheets 32 of the cells 34 to form with only the cells 34 at the particular location of the sheet 32 within the vessel 12. Now that each of the sheets 32 are configured at the desired location with the desired cell type, the specialized nutrients for the particular cell types can then be delivered to that location through the corresponding microfluidic tube 40. Particularly, each of the tubes 40 can provide different nutrients at different times and at different rates and at different locations to facilitate and optimize the tissue regeneration process within the vessel 12. Thus, the system 10 allows the various nutrients to be delivered to the generating tissue at the right time for a faster and more natural tissue regeneration process. A delivery and metering device 42, such as a syringe-type device or devices, is provided that delivers the desired amount of nutrients and other materials to the particular tubes 40 at the desired times.

In addition to providing the standing acoustic fields within the vessel 12 to generate the sheets 32 of the cells 34, the present invention also proposes simultaneously providing an electric field within the vessel 12 that provides dielectrophoretic forces to control the position and orientation of endothelial cells to provide tissue vascularization as the tissue is being regenerated. As discussed above, electric fields act on the cells to create linear cell arrays or chains. An integrated circuit 50, or some other suitable device, is provided in the base portion 16 including a system of electrodes 52 that when energized by power lines 54 creates electric fields within the vessel 12 to provide the dielectrophoretic forces in a manner that is well understood by those skilled in the art. The circuit 50 can also be provided at the other end of the vessel 12.

Endothelial cells are introduced into the vessel 12, such as, for example, through one or more of the tubes 40 at the appropriate time and at the appropriate location to be integrated into the forming tissue. As mentioned above, providing electric fields causes particles, here the endothelial cells, to be formed in a chain configuration, which is suitable to provide vascularization within the generating tissue so that the tissue is able to survive. By providing the endothelial cells to the vessel 12 while the electric field is being applied, those endothelial cells will be configured in the chain format within, between and among the several sheets 32 of the tissue cells 34 that are generating the tissue.

The foregoing discussion disclosed and describes merely exemplary embodiments of the present invention. One skilled in the art will readily recognize from such discussion and from the accompanying drawings and claims that various changes, modifications and variations can be made therein without departing from the spirit and scope of the invention as defined in the following claims.

What is claimed is:

1. A system for regenerating tissue, said system comprising:
a vessel containing a fluid suitable for enhancing tissue generation; and
an acoustic transducer providing an acoustic signal that generates standing acoustic fields in the vessel, said standing acoustic fields substantially confining cells within the fluid into at least one cell sheet to cause the cells to generate an extracellular matrix and subsequently the tissue.

2. The system according to claim 1 further comprising an electric field generating circuit for generating an electric field within the vessel that provides dielectrophoretic forces to create cellular arrays relative to the at least one cell sheet.

3. The system according to claim 2 wherein the cellular arrays generate vascularization for the tissue.

4. The system according to claim 1 further comprising at least one microfluidic channel being operable to provide sustaining tissue regeneration materials to the vessel.

5. The system according to claim 4 wherein the at least one microfluidic channel provides the cells to the vessel.

6. The system according to claim 4 wherein the at least one microfluidic channel provides cell nutrients to the vessel.

7. The system according to claim 4 wherein the at least one microfluidic channel provides endothelial cells to the vessel to provide vascularization for the tissue.

8. The system according to claim 4 wherein the at least one microfluidic channel is a plurality of microfluidic channels strategically positioned at different locations within the vessel to deliver the material at different locations in vessel.

9. The system according to clam 1 wherein the tissue is human tissue.

10. A system for regenerating human tissue, said system comprising:
a vessel containing a fluid suitable for enhancing tissue regeneration;
an acoustic transducer providing an acoustic signal to generate standing acoustic fields in the vessel, said standing acoustic fields substantially confining cells within the fluid into a plurality of cell sheets to cause the cells to regenerate tissue; and
an electric field generating circuit for generating an electric field within the vessel that provides dielectrophoretic forces to create cellular arrays relative to the plurality of cell sheets to provide vascularization for the tissue.

11. The system according to claim 10 further comprising at least one microfluidic channel being operable to provide sustaining tissue regeneration materials to the vessel.

12. The system according to claim 11 wherein the at least one microfluidic channel provides the cells to the vessel.

13. The system according to claim 11 wherein the at least one microfluidic channel provides cell nutrients to the vessel.

14. The system according to claim 11 wherein the at least one microfluidic channel provides endothelial cells to the vessel to provide vascularization.

15. The system according to claim 11 wherein the at least one microfluidic channel is a plurality of microfluidic channels strategically positioned at different locations within the vessel to deliver the material at different locations in vessel.

16. A method for regenerating tissue, said method comprising:
generating standing acoustic fields within a vessel so as to confine cells within the vessel as a plurality of tissue cell sheets at locations in the vessel that allow the tissue cell sheets to form an extra cellular matrix and regenerate the tissue; and
providing nutrients and other materials to the vessel to enhance the tissue regeneration.

17. The method according to claim 16 further comprising generating an electric field within the vessel that provides dielectrophoretic forces to create cellular arrays relative to the at least one cell sheet, where the cellular arrays generate vascularization for the tissue.

18. The method according to claim 16 further comprising providing a plurality of microfluidic channels that provide tissue sustaining regeneration materials to the vessel.

19. The method according to claim 18 wherein the plurality of microfluidic channels provide one or more of the cells, nutrients or endothelial cells.

20. A method for causing human tissue growth comprising:
suspending human cells in a fluid media; and
applying a field-induced force to the suspended cells in a manner that allows the cells to assemble into a three-dimensional structure for a period of time effective to allow the cells to form a natural extra cellular matrix.
